(19) United States
(12) Patent Application Publication
(10) Pub. No.: US 2021/0161774 A1
(43) Pub. Date: Jun. 3, 2021

(54) METHOD FOR PRODUCING SOLID POWDER COSMETIC

(71) Applicant: Tokiwa Corporation, Nakatsugawa-shi (JP)

(72) Inventor: Natsumi Wakabayashi, Kawaguchi-shi (JP)

(21) Appl. No.: 16/952,083

(22) Filed: Nov. 19, 2020

(30) Foreign Application Priority Data
Nov. 28, 2019 (JP) 2019-215400

(51) Int. Cl. A61K 8/02; A61K 8/92; A61K 8/31; A61K 8/891; A61K 8/26; A61K 8/19; A61K 8/25; A61K 8/23; A61K 8/29

(52) U.S. Cl. CPC A61K 8/0254; A61K 8/92; A61K 8/31; A61K 8/891; A61K 8/29; A61K 8/19; A61K 8/25; A61K 8/23; A61K 8/26

(57) ABSTRACT

A method for producing a solid powder cosmetic, the method including: a step of preparing a slurry containing a cosmetic base material containing an oil component and a powder component, and a volatile hydrocarbon solvent; and a step of compression-molding the slurry, in which the oil component contains an ester oil that is insoluble in the hydrocarbon solvent and that has a hydroxyl value of 40 to 85, and the powder component contains a plate-like powder.

### CROSS-REFERENCE TO RELATED APPLICATIONS

This application claims the benefit of priority from Japanese Patent Application No. 2019-215400, filed on Nov. 28, 2019, the entire contents of which are incorporated herein by reference.

### TECHNICAL FIELD

The present disclosure relates to a method for producing a solid powder cosmetic.

### BACKGROUND

Solid powder cosmetics are composed of a powder component and an oil component and are used for makeup cosmetics such as eyeshadows, blushes, and foundations. Such solid powder cosmetics require sufficient impact resistance so that they can cope with a situation of being carried. In addition, regarding a sensation when used, it is desired that saturation or color development at the time of application be high, and a cosmetic spread smoothly on the skin and firmly adhere thereto to improve long-lasting makeup properties.

Known methods for producing a solid powder cosmetic, the following methods include: a dry-type production method in which a powder component and an oil component are mixed, and filled into a mold to be pressed and molded; and a wet-type production method in which a powder component and an oil component are mixed with a solvent to form a slurry, the obtained slurry is filled into a mold and lightly pressed, and then molding is performed by drying the solvent to remove it (for example, Japanese Unexamined Patent Publication No. 2016-124839).

### SUMMARY

A solid powder cosmetic obtained by the wet-type production method is brought into a form of slurry, whereby a powder and an oil agent are uniformly dispersed and it is easy to obtain a delicate powdery quality and a moisturizing sensation when used, whereas a surface of a molded cosmetic may look whitish, that is, so-called color loss tends to occur. In the case of a cosmetic in which color loss has occurred, it is difficult to have an appearance color developed as an applied color when buyers apply the cosmetic, as well as to have the appeal of favorable saturation or color development at the time of application.

Saturation or color development of solid powder cosmetics can be improved by blending in plate-like powders according to the use purpose. For example, when a pearly sensation is to be imparted, glitter powders having plate-like powders as a base are used. In order to obtain favorable spreadability and adhesiveness at the time of application, plate-like powders such as talc are used. However, when a large amount of a plate-like powder is added to a solid powder cosmetic, impact resistance tends to deteriorate. In a composition containing a large amount of an oil component for the purpose of inhibiting this deterioration, poor molding is likely to occur.

Disclosed herein is a method for producing a solid powder cosmetic which has sufficient impact resistance and in which saturation or color development at the time of application is sufficiently high, a sensation when used (such as spreadability and adhesion to the skin) is excellent, and a color difference between a surface color of an unused part and a color of its content is small.

In order to achieve the method, the present inventors have made extensive studies and have found that it is possible to obtain a solid powder cosmetic in which all of color development and a sensation when used, color tone on a press-molded surface, and impact resistance are all favorable by using a combination of an ester oil having a specific hydroxyl value and a volatile hydrocarbon solvent that is incompatible with the ester oil as an oil component and a solvent, which are contained in the slurry, in a case of producing a solid powder cosmetic containing a plate-like powder by a wet-type production method.

In some examples, a method for producing a solid powder cosmetic may comprise: a step of preparing a slurry comprising a cosmetic base material comprising an oil component and a powder component, and a volatile hydrocarbon solvent; and a step of compression-molding the slurry, wherein the oil component comprises an ester oil (A) that is insoluble in the hydrocarbon solvent and has a hydroxyl value of 40 to 85, and the powder component comprises a plate-like powder (B).

According to the method for producing a solid powder cosmetic, it is possible to obtain a solid powder cosmetic which has sufficient impact resistance and in which saturation or color development at the time of application is sufficiently high, a sensation when used (such as spreadability and adhesion to the skin) is excellent, and a color difference between a surface color of an unused part and a color of its content is small.

From the viewpoint of achieving the above effects at a high level, the ester oil (A) may comprise a structural unit derived from hydrogenated castor oil and/or a structural unit derived from a dimer acid.

From the viewpoint of achieving the above effects at a higher level, the ester oil (A) may comprise at least one ester oil selected from the group consisting of a Polyglyceryl-2 Isostearate/Dimer Dilinoleate Copolymer, a Hydrogenated Castor Oil/Sebacic Acid Copolymer, and a hydrogenated castor oil of dimer dilinoleic acid.

The hydrocarbon solvent may comprise at least one of isoparaffin, isododecane or a mixture thereof.

A content of the powder component in the cosmetic base material may be 75 to 95 mass % with respect to a total amount of the cosmetic base material.

The plate-like powder (B) may comprise a plate-like powder coated with a metal oxide. In this case, characteristics resulting from the metal oxide coating can be imparted to the solid powder cosmetic.

A content of the ester oil (A) in the cosmetic base material may be 0.1 to 5 mass % with respect to a total amount of the cosmetic base material.

### DETAILED DESCRIPTION

In one or more embodiments, a method for producing a solid powder cosmetic comprising a step of preparing a slurry comprising a cosmetic base material containing an oil component and a powder component, and a volatile hydrocarbon solvent (hereinafter also referred to as a slurry preparation step); and a step of compression-molding the slurry (hereinafter, also referred to as a molding step).

As used herein, the term “volatile solvent” means a solvent having a boiling point of 250° C. or lower at one atmospheric pressure (101.325 kPa).

The oil component and the powder component which are contained in the cosmetic base material constituting the solid powder cosmetic will be described.

(Oil Component)

As the oil component, components usually used in cosmetics can be blended in, and for example, solid oils and other oil agents oil can be used. The oil component may be used alone, or two or more kinds thereof may be used in combination.

Examples of the solid oil include hydrocarbons such as paraffin wax, microcrystalline wax, and polyethylene; plant-derived fats and oils such as hardened castor oil, hydrogenated jojoba oil, carnauba wax, and rice wax; esters such as glyceryl tribehenate and a cholesterol fatty acid ester; higher fatty acids such as stearic acid and behenic acid; higher alcohols such as stearyl alcohol and behenyl alcohol; silicones such as an alkyl-modified silicone and an acrylic-modified silicone; and sugar fatty acid esters such as dextrin palmitate and inulin stearate. These may be used alone, or two or more kinds thereof may be used in combination.

As the oil agent other than the solid oil, a paste oil or a liquid oil can be used. Examples of the paste oil include vaseline, hexa(hydroxystearic acid/stearic acid/rosin acid)dipentaerythrityl, tetra(hydroxystearic acid/isostearic acid)dipentaerythrityl, dipentaerythrityl hexahydroxystearate, tri(caprylic acid/capric acid/myristic acid/stearic acid)glyceryl, isostearic acid hydrogenated castor oil, phytosteryl oleate, sucrose tetrastearate triacetate, and hexa(oleic acid/palmitic acid/stearic acid)sucrose. These may be used alone, or two or more kinds thereof may be used in combination.

Examples of the liquid oil include non-volatile hydrocarbon oils such as liquid paraffin, squalane, hydrogenated polyisobutene, and hydrogenated polydecene; ester oils such as diisostearyl malate, polyglyceryl triisostearate, dipentaerythrityl pentaisostearate, and trimethylolpropane triisostearate; vegetable oils such as castor oil; higher alcohols; higher fatty acids; and silicone oils such as methylpolysiloxane, methylphenylpolysiloxane, octamethylcyclotetrasiloxane, decamethylcyclopentasiloxane, dodecamethylcyclohexasiloxane, stearoxymethylpolysiloxane, diphenylsiloxyphenyltrimethicone, and fluorine-modified silicone. These may be used alone, or two or more kinds thereof may be used used in combination.

In one or more embodiments, a content of the oil component in the cosmetic base material of the present embodiment may be 5 to 20 mass % with respect to a total amount of the cosmetic base material, and it may be 7 to 18 mass %, 8 to 16 mass %, or 10 to 15 mass %. Impact resistance and anti-caking can be improved by employing the content of the oil component in one of the increasingly narrower ranges disclosed above, such as 7 to 18 mass %.

The cosmetic base material may comprise as an oil component, an ester oil that is insoluble in the volatile hydrocarbon solvent and that has a hydroxyl value of 40 to 85 (hereinafter also referred to as an ester oil (A)). The ester oil (A) may be used alone, or two or more kinds thereof may be used in combination.

As used herein, the ester oil insoluble in the hydrocarbon solvent, (being incompatible with each other) means one in which white turbidity or separation is observed when 0.5 g of the ester oil and 5 g of the hydrocarbon solvent are added at 25° C., mixed using a stirring rod, and left standing for 15 minutes, and then the state is visually observed. When two or more volatile hydrocarbon solvents are to be contained in the slurry, an ester oil which is confirmed to be insoluble in the mixture according to the above-described method can be combined for use.

The hydroxyl value of the ester oil means a value measured in accordance with a “Standard Oil and Fat Analysis Test Method” (established by Japan Oil Chemists' Society).

The ester oil (A) may have a hydroxyl value of 40 to 85, 45 to 80, or 45 to 75.

As the ester oil (A), an ester oil comprising a structural unit derived from hydrogenated castor oil (hardened castor oil) and/or a structural unit derived from a dimer acid may be used from the viewpoint of further improving all of color development and a sensation when used, color tone on a press-molded surface, and impact resistance of the solid powder cosmetic.

As the ester oil comprising a structural unit derived from a dimer acid, it is possible to use a dibasic acid ester obtained by polymerization of two molecules of unsaturated fatty acid. Examples thereof include: esters of dimer dilinoleic acid, dimer dilinoleic acid, or dimer dioleic acid; and hydrogenated products of the foregoing. Examples of an ester portion of the dimer acid ester include: an alkyl groups or an alkenyl groups derived from higher alcohols such as oleyl alcohol, stearyl alcohol, behenyl alcohol, lauryl alcohol, and linoleyl alcohol; from polyhydric alcohols such as diglycerin and glycerin; or from dimer diols in which two molecules of unsaturated alcohols, such as dimer dilinoleyl alcohol, are polymerized.

As the ester oil comprising a structural unit derived from a dimer acid, a Hydrogenated Castor Oil/Sebacic Acid Copolymer may be used. This copolymer can be generated by, for example, a condensation reaction of diglycerin, isostearic acid, and a hydrogenated dimer acid, and it is also possible to use commercially available products such as “Hailucent ISDA” (trade name, manufactured by KOKYU ALCOHOL KOGYO CO., LTD.).

As the ester oil comprising a structural unit derived from a hydrogenated castor oil, an oligoester of hydrogenated castor oil and a saturated dibasic acid may be used. Examples of the saturated dibasic acid include succinic acid, adipic acid, azelaic acid, sebacic acid, octadecamethylenedicarboxylic acid, and eicosadicarboxylic acid.

As the ester oil comprising a structural unit derived from hydrogenated castor oil, a (hydrogenated castor oil/sebacic acid) copolymer may further be used. The copolymer can employ a commercially available product such as “Crodabond CSA-LQ-(JP)” (trade name, manufactured by Croda Japan KK).

As the ester oil comprising a structural unit derived from hydrogenated castor oil and a structural unit derived from a dimer acid, an oligomer of hydrogenated castor oil and a dimer acid may be used.

As the oligomer of hydrogenated castor oil and a dimer acid, commercially available hydrogenated castor oils of dimer dilinoleic acid may be used. Examples of the commercially available product include “Risocast DA-L” and “Risocast DA-H” (trade name, manufactured by KOKYU ALCOHOL KOGYO CO., LTD.).

The ester oil (A) may be in the form of a paste or one having a viscosity of 10,000 mPa·s or more at 25° C. When the viscosity of an ester oil is 10,000 to 100,000 mPa·s, it is measured at a rotational speed of 10 rpm using a BH type viscometer and a No. 6 rotor. When the viscosity of an ester oil is 100,000 to 350,000 mPa·s, it is measured under conditions of 25° C. and a rotational speed of 10 rpm using a BH type viscometer and a No. 7 rotor. The viscometer and rotor may be products of TOKI SANGYO CO., LTD. A viscosity of the ester oil (A) may be 10,000 to 200,000 mPa·s, or 40,000 to 100,000 mPa·s.

From the viewpoint of further improving all of color development and a sensation when used, color tone on a press-molded surface, and impact resistance of the solid powder cosmetic, the cosmetic base material according to one or more embodiments may comprise, as the ester oil (A), at least one ester oil selected from the group consisting of a (polyglyceryl-2 isostearate/dimer dilinoleic acid) copolymer, a (hydrogenated castor oil/sebacic acid) copolymer, and a hydrogenated castor oil of dimer dilinoleic acid. Furthermore, when the cosmetic base material comprises at least one of these ester oils, the volatile hydrocarbon solvent may comprise at least one of isoparaffin, isododecane or a mixture thereof.

In one or more embodiments, a content of the ester oil (A) in the cosmetic base material may be 0.1 to 5 mass %, 0.5 to 4 mass %, 1 to 3 mass %, or 1.5 to 3 mass % with respect to a total amount of the cosmetic base material. Impact resistance and a sensation when used can be improved by employing the content of the ester oil (A) in one of the increasingly narrower ranges disclosed above, such as 0.1 to 5 mass %.

In one or more embodiments, a content ratio of the ester oil (A) in the oil component contained in the cosmetic base material is such that a mass ratio [ester oil (A)]/[oil component] of the ester oil (A) with respect to a total amount of the oil component may be 0.1/20 to 1/1, 0.5/20 to 4/7, 1/16 to 3/8, or 1/10 to 1/5. Impact resistance and a sensation when used can be improved by employing the mass ratio [ester oil (A)]/[oil component] of the ester oil (A) with respect to a total amount of the oil component in one of the increasingly narrower ranges disclosed above, such as 0.1/20 to 1/1.

In one or more embodiments, a content ratio of the ester oil (A) and the volatile hydrocarbon solvent in the slurry of the present embodiment is such that a mass ratio [ester oil (A)]/[volatile hydrocarbon solvent] of the ester oil (A) with respect to the volatile hydrocarbon solvent may be 0.1/80 to 5/10, 0.5/75 to 4/15, or 1/70 to 3/20. Saturation and color development of a press-molded surface can be improved by employing the mass ratio [ester oil (A)]/[volatile hydrocarbon solvent] of the ester oil (A) with respect to the volatile hydrocarbon solvent in one of the increasingly narrower ranges disclosed above, such as 0.1/80 to 5/10.

(Powder component)

As the powder component, any powder that is usually used in cosmetics can be used. Examples thereof include extender powders, white pigments, and coloring pigments. In one or more embodiments, the powder may have a spherical, plate-like, needle-like shape, or smoke-like shape, and may have a particle structure such as a fine particle, a particle size of a pigment grade, a porous structure, and a non-porous structure. The powder component may be used alone, or two or more kinds thereof may be used in combination.

Examples of the powder component include extender pigments such as mica, synthetic mica, sericite, talc, kaolin, silicon carbide, barium sulfate, bentonite, smectite, aluminum oxide, silica, magnesium oxide, zirconium oxide, magnesium carbonate, calcium carbonate, chromium oxide, and magnesium aluminum hydroxide; ultraviolet scattering agents such as fine particle titanium oxide and fine particle zinc oxide; organic powders such as nylon powder, polymethylmethacrylate powder, acrylonitrile-methacrylic acid copolymer powder, vinylidene chloride-methacrylic acid copolymer, polyethylene powder, polystyrene powder, organopolysiloxane elastomer powder, polymethylsilsesquioxane powder, urethane powder, wool powder, silk powder, cellulose powder, and N-acyllysine powder; composite powders such as fine particle titanium oxide-coated mica titanium, fine particle titanium oxide-coated nylon, barium sulfate-coated mica titanium, titanium oxide-containing silica, and zinc oxide-containing silica; and soaps formed of metals such as magnesium stearate, zinc myristate, aluminum stearate, and calcium stearate.

Examples of the coloring pigment include: inorganic coloring pigments such as red iron oxide, yellow iron oxide, black iron oxide, cobalt oxide, chromium oxide, ultramarine blue, navy blue, titanium oxide, and zinc oxide; organic coloring pigments such as red 228, red 226, blue 404, red 202, and yellow 4 aluminum lake; pearl pigments such as mica titanium, fine particle titanium oxide-coated mica titanium, barium sulfate-coated mica titanium, fish scale foil, bismuth oxychloride, and aluminum flakes; and natural pigments such as carmine and safflower.

These powder components may be powders treated for hydrophobicity from the viewpoint of color development and adhesion. Examples of the hydrophobic treatments include treatment with higher fatty acids, metal soaps, oils and fats, waxes, silicone compounds, fluorine compounds, surfactants, and dextrin fatty acid esters.

In the cosmetic base material according to one or more embodiments, the powder component may comprise a plate-like powder (B) from the viewpoint that the cosmetic spreads smoothly on the skin and firmly adheres thereto to improve long-lasting makeup properties.

In one or more embodiments, an average particle size of the plate-like powder may be 0.1 to 300.0 μm, 1.0 to 200 μm, or 3.0 to 100 μm. Similarly, an aspect ratio of the plate-like powder may be 8 to 400, 10 to 300, or 15 to 200. Color development and a sensation when used can be improved by employing the average particle size of the plate-like powder in one of the increasingly narrower ranges disclosed above, such as 8 to 400. The average particle size of the plate-like powder means a 50% median size measured by a laser diffraction method. The aspect ratio means a ratio of average particle size/average thickness.

As the plate-like powder, for example, it is possible to use talc, mica, synthetic mica, sericite, silicic acid anhydride, kaolin, calcium carbonate, alumina, or glass. The plate-like powder may be a coating-treated one.

In one or more embodiments, the cosmetic base material may comprise a plate-like powder coated with a metal oxide from the viewpoint of improving saturation or color development of the solid powder cosmetic. In this case, characteristics (for example, a pearly sensation) resulting from the metal oxide coating can be imparted to the solid powder cosmetic. Examples of the plate-like powders coated with a metal oxide include glitter powder, iron oxide-coated titanium mica, iron oxide-coated mica, iron oxide-coated talc, zinc oxide-coated mica, and zinc oxide-coated talc. In the glittering powder, the plate-like powder which is a base may be alumina, glass, or mica.

Alumina and glass have a lower oil absorption than mica.

When they are mixed with an oil component, generation of caking and deterioration in removing makeup easily occur. When a blending amount of the oil component is reduced in order to avoid this occurrence, it is difficult for powders to be bonded to each other, and impact resistance tends to deteriorate. Furthermore, during the production of solid powder cosmetics by a wet-type method, in a case where an oil component is mixed with a volatile solvent, imparts viscosity to the entire solvent, and results in a too high viscosity, the solvent is insufficiently sucked at the time of pressing, which tends to cause defective compression-molding. When a blending amount of the oil component is then reduced in this case, bonding of powders is insufficient and the impact resistance deteriorates, as described above.

In contrast, according to the method for producing a solid powder cosmetic described herein, even in a case where the glittering powder is blended in, it is possible to obtain a solid powder cosmetic which has sufficient impact resistance and in which saturation or color development at the time of application is sufficiently high, a sensation when used such as spreadability and adhesion to the skin is excellent, and a color difference between a surface color of an unused part and a color of its content is small. It is possible to prevent insufficient suction of the solvent and poor compression-molding caused by an increase in the viscosity of the solvent by blending the insoluble ester oil (A) into the volatile hydrocarbon solvent. Furthermore, since the ester oil (A) is insoluble in the volatile hydrocarbon solvent, it easily remains in a cosmetic even when the solvent is sucked, bonding between powders can be assured without blending in a large amount of the oil component, and as a result, impact resistance can be improved. In addition, the ester oil (A) having a hydroxyl value in a specific range inhibits dispersion of a pigment in the solvent, and thereby, color loss during pressing is reduced.

In one or more embodiments, a content of the powder component in the cosmetic base material of the present embodiment may be 75 to 95 mass %, 80 to 95 mass %, or 80 to 90 mass %, with respect to a total amount of the cosmetic base material. Saturation or color development at the time of application, and smooth spreading on the skin can be improved by employing the content of the powder component in the cosmetic base material of the present embodiment in one of the increasingly narrower ranges disclosed above, such as 75 to 95 mass %.

In one or more embodiments, a content of the plate-like powder (B) in the cosmetic base material of the present embodiment may be 3 to 90 mass %, 20 to 87 mass %, or 4 to 85 mass %, with respect to a total amount of the cosmetic base material. The cosmetic can spreads more smoothly on the skin and more firmly adheres thereto to improve long-lasting makeup properties by employing the content of the plate-like powder (B) in the cosmetic base material in one of the increasingly narrower ranges disclosed above, such as 3 to 90 mass %.

In one or more embodiments, a mass ratio [(A)/(B)] of the ester oil (A) and the plate-like powder (B) in the cosmetic base material may be 0.1/90 to 5/75, or 1/80 to 3/90. Impact resistance and usability can be improved by employing the mass ratio [(A)/(B)] of the ester oil (A) and the plate-like powder (B) in the cosmetic base material in one of the increasingly narrower ranges disclosed above, such as 0.1/90 to 5/75.

In a case where the cosmetic base material further contains a plate-like powder (B′) coated with a metal oxide, a mass ratio [(B′)/(B)] of the plate-like powder (B) and the plate-like powder (B′) coated with a metal oxide may be 3/95 to 1/1, or 5/90 to 1/2. Furthermore, a mass ratio [(A)/(B′)] of the ester oil (A) and the plate-like powder (B′) coated with a metal oxide may be 0.5/95 to 1/1, or 1/90 to 1/2.

In one or more embodiments, a total content of the oil component and the powder component in the cosmetic base material of the present embodiment may be 85 to 100 mass %, 87 to 99 mass %, or 90 to 98 mass %, with respect to a total amount of the cosmetic base material. The cosmetic can spreads more smoothly on the skin and more firmly adheres thereto to improve long-lasting makeup properties by employing the total content of the oil component and the powder component in the cosmetic base material of the present embodiment in one of the increasingly narrower ranges disclosed above, such as 85 to 100 mass %.

In addition to the above-mentioned components, the cosmetic base material may contain components usually used in cosmetics, for example, preservatives, antioxidants, pigments, thickeners, pH adjusters, fragrances, ultraviolet absorbers, moisturizers, chelating agents, and antiphlogistic drugs, surfactants.

(Volatile hydrocarbon solvent) The volatile hydrocarbon solvent contained in the slurry will be described.

As described above, the volatile hydrocarbon solvent is in an incompatible with the ester oil (A). The volatile hydrocarbon solvent can be appropriately selected, depending on the ester oil (A) blended therein, it may comprise at least one of isoparaffin, isododecane or a mixture thereof. Light liquid isoparaffin can be used as isoparaffin.

(Slurry preparation step)

As an example of method of preparing a slurry, there is mentioned a method of adding a volatile hydrocarbon solvent to a cosmetic base material containing the above-described blending amounts of an oil component and a powder component and mixing them.

The cosmetic base material may be prepared by a method including a step of obtaining a first mixture in which a powder component is mixed, a step of obtaining a second mixture in which an oil component is mixed, and a step of mixing the first mixture and the second mixture.

The step of obtaining the first mixture can be performed using, for example, a super mixer, or a Henschel mixer, and pulverization may be performed using an atomizer, if necessary.

The step of obtaining the second mixture can be performed using, for example, a disper, or a homomixer, and the oil component can be mixed while heating at 60° C. to 80° C., or 60° C. to 70° C.

The step of mixing the first mixture and the second mixture can be performed using, for example, a super mixer, or a Henschel mixer, and pulverization may be performed using an atomizer or the like, if necessary.

The cosmetic base material and the volatile hydrocarbon solvent can be mixed by, for example, a method of kneading using a kneader or a universal stirrer. Furthermore, they can be mixed while heating if necessary.

In one or more embodiments, a blending ratio of the cosmetic base material and the volatile hydrocarbon solvent in the slurry is such that a mass ratio of the cosmetic base material:the volatile hydrocarbon solvent is 100:10 to 100:80; and a mass ratio thereof may be 100:20 to 100:60 from the viewpoint of moldability.

In one or more embodiments, the slurry may contain a dispersion medium other than the above-mentioned volatile hydrocarbon solvent as long as the intended effects are not impaired. Examples of the dispersion media include volatile solvents such as water, isopropyl alcohol, acetone, and ethyl alcohol.

(Molding Step)

In the step of compression-molding the slurry the slurry obtained above is, for example, defoamed as necessary, filled into a predetermined container, compression-molded by suction compression-molding, and then appropriately dried with a dryer.

Examples of the predetermined container include an inner tray such as a metal tray and a resin tray.

A solid powder cosmetic may be obtained through the above steps. The solid powder cosmetic can have the same composition as the above-mentioned cosmetic base material.

The solid powder cosmetic is suitable as a makeup cosmetic such as foundations, face colors, eyeshadows, eyebrow cosmetics, and blushes.

Hereinafter, details of additional example embodiments will be described with reference to comparative examples. Numerical values in tables each show a content (mass %) on the basis of a total amount of a cosmetic base material (a total of components other than a volatile solvent). The volatile solvent represents a ratio (parts by mass) to 100 parts by mass of the total amount of the cosmetic base material (a total of components other than the volatile solvent).

An evaluation method used in each example will be described.

(1) Impact Resistance

A sample of each solid powder cosmetic was placed in a direction in which its contents were facing upward, and a state after having been dropped 5 times on a P tile from a height of 50 cm was visually observed. Evaluation was performed in 4 stages according to the following evaluation criteria to rate each of the samples, and an average point (n=5) of scores of all panels was determined according to the following criteria.

[Score: Evaluation Criteria]
4 points: no change
3 points: some floating, chipping, and cracking occurred, but not regarded as problems
2 points: partly floated, chipped, or cracked
1 point: entirely floated, chipped, and cracked

[Determination Criteria (Average Point of Scores)]
A: 3.5 or more
B: 3.0 or more and less than 3.5
C: 2.0 or more and less than 3.0
D: less than 2.0

(2) Color Difference

A cosmetic surface was repeatedly rubbed off 30 times with a cosmetic tip, and a color difference ΔE* between a surface color of an used part and a surface color of an unused part was measured using a colorimeter CR-400 (manufactured by Konica Minolta, Inc.). It was determined according to the following evaluation criteria.

[Evaluation Criteria]

A: ΔE*<5.0

B: 5.0≤ΔE*<7.0

C: 7.0≤ΔE*<9.0

D: 9.0≤ΔE*

(3) Degree of Saturation (Transparency) or Color Development, and Sensation when Used

twenty panelists specializing in cosmetics were asked to use the solid powder cosmetics of examples and comparative examples. Individual evaluation was performed on “a degree of saturation (transparency) or color development,” and “a degree of spreadability” and “adhesion” as sensations when used in 5 stages according to the following evaluation criteria to rate each of the solid powder cosmetics. An average point of scores of the all panelists was determined according to the following criteria.

[Score: Evaluation Criteria]
5 points: very favorable
4 points: favorable
3 points: moderate
2 points: slightly poor
1 point: poor

[Determination Criteria (Average Point of Scores)]
A: 4 or more
B: 3 or more and less than 4
C: 2 or more and less than 3
D: less than 2

### Examples 1 to 9 and Comparative Examples 1 to 5

Eye shadows having the compositions shown in Tables 1 and 2 were prepared by the production method and evaluated, as described above. The results are also shown in Tables 1 and 2.

<Production Method>

Powder components were mixed using a Henschel mixer, an oil component separately heated and mixed at 60° C. to 80° C. was added thereto, and the mixture was stirred using a Henschel mixer to prepare a cosmetic base material. A predetermined amount of a volatile solvent shown in the table was added and kneaded to form a slurry. An inner tray (i.2., metal tray) was filled with this slurry, suction pressing and drying were applied thereto, whereby performed, and thereby an eye shadow sample was produced.

TABLE 1

| | | Example | Example | Example | Example | Example | Example | Example | Example | Example |
|---|---|---|---|---|---|---|---|---|---|---|
| | | 1 | 2 | 3 | 4 | 5 | 6 | 7 | 8 | 9 |
| Cosmetic | Ester oil-1 | 2.00 | — | 0.50 | 4.00 | 2.00 | 2.00 | 2.00 | 2.00 | 3.00 |
| base | Ester oil-2 | — | 2.00 | — | — | — | — | — | — | — |
| material | Diphenylsiloxyphenyltri- | 6.00 | 6.00 | 6.00 | 6.00 | 6.00 | 6.00 | 6.00 | 6.00 | 6.00 |
| (mass %) | methicone | | | | | | | | | |
| | Hydrogenated polydecene | 4.45 | 4.45 | 4.45 | 4.45 | 4.45 | 4.45 | 4.45 | 4.45 | 4.45 |
| | Dimethicone (5,000 cs) | 2.00 | 2.00 | 3.50 | — | 2.00 | 2.00 | 2.00 | 2.00 | 2.00 |
| | Antioxidant | 0.05 | 0.05 | 0.05 | 0.05 | 0.05 | 0.05 | 0.05 | 0.05 | 0.05 |
| | Preservative | 0.30 | 0.30 | 0.30 | 0.30 | 0.30 | 0.30 | 0.30 | 0.30 | 0.30 |
| | Alumina base glittering | 35.00 | 35.00 | 35.00 | 35.00 | — | — | 35.00 | 5.00 | — |
| | powder | | | | | | | | | |
| | Glass base glittering | — | — | — | — | 35.00 | — | — | — | — |
| | powder | | | | | | | | | |
| | Mica base glitter powder | — | — | — | — | — | 35.00 | — | 10.00 | — |
| | Black iron oxide-coated | — | — | — | — | — | — | — | — | 15.00 |
| | mica titanium | | | | | | | | | |
| | Aluminium stearate | 0.50 | 0.50 | 0.50 | 0.50 | 0.50 | 0.50 | 0.50 | 0.50 | 0.50 |
| | barium sulfate | 2.00 | 2.00 | 2.00 | 2.00 | 2.00 | 2.00 | 2.00 | 2.00 | 2.00 |
| | Alumina | 3.00 | 3.00 | 3.00 | 3.00 | 3.00 | 3.00 | 3.00 | 3.00 | 3.00 |
| | Synthetic phlogopite | 15.00 | 15.00 | 15.00 | 15.00 | 15.00 | 15.00 | 15.00 | 15.00 | 10.00 |
| | Talc | 20.00 | 20.00 | 20.00 | 20.00 | 20.00 | 20.00 | 20.00 | 20.00 | 15.00 |
| | Mica | 5.70 | 5.70 | 5.70 | 5.70 | 5.70 | 5.70 | 5.70 | 9.70 | 26.70 |
| | Coloring pigment-1 | 4.00 | 4.00 | 4.00 | 4.00 | 4.00 | 4.00 | 4.00 | 20.00 | 12.00 |
| | Total | 100.00 | 100.00 | 100.00 | 100.00 | 100.00 | 100.00 | 100.00 | 100.00 | 100.00 |
| Volatile | Light liquid isoparaffin | 30.00 | 30.00 | 30.00 | 30.00 | 30.00 | 40.00 | — | 30.00 | 40.00 |
| hydrocarbon | Isododecane | — | — | — | — | — | — | 30.00 | — | — |
| solvent | | | | | | | | | | |
| (part by | | | | | | | | | | |
| mass) | | | | | | | | | | |
| Compatibility of ester oil and volatile | | Insoluble | Insoluble | Insoluble | Insoluble | Insoluble | Insoluble | Insoluble | Insoluble | Insoluble |
| hydrocarbon solvent | | | | | | | | | | |
| Evaluation | Impact resistance | A | A | B | A | A | B | A | A | A |
| | Color difference | A | A | A | A | B | B | A | B | A |
| | Saturation (transparency) or | A | A | A | A | A | B | A | B | A |
| | color development | | | | | | | | | |
| | Sensation when used | A | B | B | B | B | B | A | B | B |
| | (spreadability and adhesion) | | | | | | | | | |

TABLE 2

| | | Comparative | Comparative | Comparative | Comparative | Comparative |
|---|---|---|---|---|---|---|
| | | Example 1 | Example 2 | Example 3 | Example 4 | Example 5 |
| Cosmetic | Ester oil-3 | 2.00 | — | — | 2.00 | 3.00 |
| base | Ester oil-4 | — | 2.00 | — | — | |
| material | Ester oil-5 | — | — | 2.00 | — | — |
| (part by | Diphenylsiloxy- | 6.00 | 6.00 | 6.00 | 6.00 | 6.00 |
| mass) | phenyltrimethicone | | | | | |
| | Hydrogenated polydecene | 4.45 | 4.45 | 4.45 | 4.45 | 4.45 |
| | Dimethicone (5,000 cs) | 2.00 | 2.00 | 2.00 | 2.00 | 2.00 |
| | Antioxidant | 0.05 | 0.05 | 0.05 | 0.05 | 0.05 |
| | Preservative | 0.30 | 0.30 | 0.30 | 0.30 | 0.30 |
| | Alumina base | 35.00 | 35.00 | 35.00 | — | — |
| | glittering powder | | | | | |
| | Glass base glittering | — | — | — | — | — |
| | powder | | | | | |
| | Mica base glitter powder | — | — | — | 35.00 | — |
| | Black iron oxide-coated | — | — | — | — | 15.00 |
| | mica titanium | | | | | |
| | Aluminium stearate | 0.50 | 0.50 | 0.50 | 0.50 | 0.50 |
| | barium sulfate | 2.00 | 2.00 | 2.00 | 2.00 | 2.00 |
| | Alumina | 3.00 | 3.00 | 3.00 | 3.00 | 3.00 |
| | Synthetic phlogopite | 15.00 | 15.00 | 15.00 | 15.00 | 15.00 |
| | Talc | 20.00 | 20.00 | 20.00 | 20.00 | 15.00 |
| | Mica | 5.70 | 5.70 | 5.70 | 5.70 | 26.70 |
| | Coloring pigment-1 | 4.00 | 4.00 | 4.00 | 4.00 | 12.00 |
| | Total | 100.00 | 100.00 | 100.00 | 100.00 | 100.00 |
| Volatile | Light liquid isoparaffin | 30.00 | 30.00 | 30.00 | 30.00 | 30.00 |
| hydrocarbon | | | | | | |
| solvent | | | | | | |
| (part by mass) | | | | | | |
| Compatibility of ester oil and | | Insoluble | Compatible | Compatible | Insoluble | Insoluble |
| volatile hydrocarbon solvent | | | | | | |
| Evaluation | Impact resistance | D | D | D | C | C |
| | Color difference | D | D | D | C | C |
| | Saturation (transparency) | C | C | C | D | D |
| | or color development | | | | | |
| | Sensation when used | C | C | C | D | D |
| | (spreadability and | | | | | |
| | adhesion) | | | | | |

Details of each component in Tables 1 and 2 are as follows.

[Oil Component]

Ester oil-1: “Hailucent ISDA” (manufactured by KOKYU ALCOHOL KOGYO CO., LTD., trade name, a (polyglyceryl-2 isostearate-2/dimer dilinoleic acid) copolymer) with a hydroxyl value of 55 and a viscosity of 52,000 mPa·s

Ester oil-2: “Crodamol CWS-S” (manufactured by Croda Japan KK, trade name, a (hydrogenated castor oil/sebacic acid) copolymer), with a hydroxyl value of 59 and a viscosity of 57,500 mPa·s

Ester oil-3: “Cosmol 168ARV” (manufactured by The Nisshin OilliO Group, Ltd., trade name, hexa(hydroxystearic acid/stearic acid/rosin acid)dipentaerythrityl) with a hydroxyl value of 90 to 110 (catalog values) in a paste form (viscosity could not be measured)

Ester oil-4: “Plandool-G” (manufactured by Nippon Fine Chemical, trade name, dimer dilinoleic acid dimer dilinoleyl bis(behenyl/isostearyl/phytosteryl)) with a hydroxyl value of 25 or less (catalog values) in a paste form (viscosity could not be measured)

Ester oil-5: “Cosmol 43N” (manufactured by The Nisshin OilliO Group, Ltd., trade name, polyglyceryl triisosterate-2) with a hydroxyl value of 30 to 50 (catalog values), and a viscosity of 22,500 mPa·s

[Powder Component]

Alumina base glittering powder: “Mirinae A-7001K Splendor Gold” (manufactured by CQV Co., Ltd., trade name, particle size: 15 to 19 μm) Glass base glittering powder: “Astral Effect Gold Shimmer” (manufactured by Croda Japan KK, particle size: 10 to 40 μm)

Mica base glitter powder: “Timiron Super Gold” (manufactured by Merck KK, particle size: 10 to 60 μm)

Black iron oxide-coated mica titanium: “Colorona Mica Black” (Merck KK, particle size: 18 to 25 μm)

Coloring pigment-1, coloring pigment-2, and coloring pigment-3: appropriate amounts of red iron oxide, yellow iron oxide, black iron oxide, and red 226 were contained to make up the respective coloring pigments.

[Volatile Solvent]

Light liquid paraffin: “IP Solvent 1620MU” (manufactured by Idemitsu Kosan Co., Ltd., trade name, boiling point: 166° C.)

(Measurement of Hydroxyl Value of Ester Oil)

A hydroxyl value of the ester oil was measured according to the “Standard Oil and Fat Analysis Test Method” (established by Japan Oil Chemists' Society).

(Measurement of Viscosity of Ester Oil)

A viscosity of the ester oil at 25° C. was measured using a BH type viscometer (manufactured by TOKI SANGYO CO., LTD). When a viscosity of the ester oil was 10,000 to 100,000 mPa·s, it was measured at a rotation speed of 10 rpm using a BH type viscometer and a rotor: No. 6. When a viscosity of the ester oil was 100,000 to 350,000 mPa·s, it was measured at a condition of a rotational speed of 10 rpm using a BH type viscometer and a rotor: No. 7.

(Compatibility of Ester Oil and Volatile Solvent)

Regarding ester oils and volatile solvents shown in the tables, 0.5 g of an ester oil and 5 g of a hydrocarbon solvent were put in a screw tube at 25° C., mixed using a stirring rod, and left standing for 15 minutes, and then the state was visually observed. The tables show a case in which white turbidity or separation was observed as “Insoluble,” and a case of transparency as “Compatible.”

As shown in Tables 1 and 2, “impact resistance,” “color difference,” “saturation (transparency) or color development,” “sensation when used (spreadability and adhesion)” of the solid powder cosmetics obtained in Examples 1 to 9 were evaluated as “A” or “B”.

### Example 10: Blush

| | (composition |
|---|---|
| | ratio |
| (Component) | (mass %)) |
| 1. Preservative | 0.60 |
| 2. Aluminium stearate | 1.00 |
| 3. Barium sulfate | 3.00 |
| 4. Alumina | 5.00 |
| 5. Synthetic phlogopite | 20.00 |
| 6. Talc | 30.00 |
| 7. Mica | 24.40 |
| 8. Coloring pigment-2 | 0.95 |
| 9. Alumina base glitter powder | 7.00 |
| 10. Dimethicone (6 cs) | 3.00 |
| 11. Ester oil-1 | 5.00 |
| 12. Antioxidant | 0.05 |
| 13. Light liquid isoparaffin | 35.00 |

Details of the above components are the same as those described above.

<Production Method>

Powder components were mixed using a Henschel mixer, an oil component separately heated and mixed at 60° C. to 80° C. was added thereto, and the mixture was stirred using a Henschel mixer to prepare a cosmetic base material. A predetermined amount of a volatile solvent shown in the table was added and kneaded to form a slurry. An inner tray (i.e., metal tray) was filled with this slurry, and suction pressing and drying were applied thereto, whereby a blush sample was produced.

<Evaluation>

The obtained blush sample was evaluated in the same manner as above. It was confirmed that the sample was evaluated as follows: “impact resistance” of “A,” “color difference” of “A,” “saturation (transparency) or color development” of “A,” and “sensation when used (spreadability and adhesion)” of “B.”

### Example 11: Highlighter

| | (composition |
|---|---|
| | ratio |
| (Component) | (mass %)) |
| 1. Preservative | 0.60 |
| 2. Aluminium stearate | 1.00 |
| 3. Barium sulfate | 3.00 |
| 4. Alumina | 5.00 |
| 5. Synthetic phlogopite | 20.00 |
| 6. Talc | 10.00 |
| 7. Mica | 3.70 |
| 8. Coloring pigment-3 | 0.70 |
| 9. Alumina base glittering powder | 45.00 |
| 10. Diphenylsiloxyphenyltrimethicone | 5.00 |
| 11. Hydrogenated polydecene | 3.95 |
| 12. Dimethicone (5000 cs) | 2.00 |
| 13. Ester oil-6 | 3.00 |
| 14. Antioxidant | 0.05 |
| 15. Light liquid isoparaffin | 35.00 |

Details of the above components are the same as those already described above except the component shown below.

Ester oil-6: “Risocast DA-L” (manufactured by KOKYU ALCOHOL KOGYO CO., LTD., trade name, a hydrogenated castor oil of dimer dilinoleic acid) with a hydroxyl value of 78 and a viscosity of 118,000 mPa·s

The compatibility of the ester oil-6 and light liquid isoparaffin was evaluated in the same manner as above, and the ester was insoluble in light liquid isoparaffin.

<Production Method>

Powder components were mixed using a Henschel mixer, an oil component separately heated and mixed at 60° C. to 80° C. was added thereto, and the mixture was stirred using a Henschel mixer to prepare a cosmetic base material. A predetermined amount of a volatile solvent shown in the table was added and kneaded to form a slurry. An inner tray (i.e., metal tray) was filled with this slurry and suction pressing and drying were applied thereto, whereby a highlighter sample was produced.

<Evaluation>

The obtained highlighter sample was evaluated in the same manner as above. It was confirmed that the sample was evaluated as follows: “impact resistance” as “B,” “color difference” as “A,” “saturation (transparency) or color development” as “A,” and “sensation when used (spreadability and adhesion)” as “A.”

What is claimed is:

1. A method for producing a solid powder cosmetic, the method comprising:
preparing a slurry comprising:
a cosmetic base material comprising an oil component and a powder component, and a volatile hydrocarbon solvent; and
compression-molding the slurry,
wherein the oil component comprises an ester oil that is insoluble in the hydrocarbon solvent and that has a hydroxyl value of 40 to 85, and
the powder component comprises a plate-like powder.

2. The method for producing a solid powder cosmetic according to claim 1,
wherein the ester oil comprises at least one structural unit selected from the group consisting of a structural unit derived from hydrogenated castor oil and a structural unit derived from a dimer acid.

3. The method for producing a solid powder cosmetic according to claim 1,
wherein the ester oil comprises at least one ester oil selected from the group consisting of a Polyglyceryl-2 Isostearate/Dimer Dilinoleate Copolymer, a Hydrogenated Castor Oil/Sebacic Acid Copolymer, and a dimer dilinoleic acid hydrogenated castor oil, and
wherein the hydrocarbon solvent comprises at least one of isoparaffin or isododecane.

4. The method for producing a solid powder cosmetic according to claim 1,
wherein a content of the powder component in the cosmetic base material is 75 to 95 mass % with respect to a total amount of the cosmetic base material.

5. The method for producing a solid powder cosmetic according to claim 1,
wherein the plate-like powder comprises a plate-like powder coated with a metal oxide.

6. The method for producing a solid powder cosmetic according to claim 1,
wherein a content of the ester oil in the cosmetic base material is 0.1 to 5 mass % with respect to a total amount of the cosmetic base material.
